(19) United States
(12) Patent Application Publication
(10) Pub. No.: US 2018/0271442 A1
(43) Pub. Date: Sep. 27, 2018

(54) Wearable Device Having Physiological and Environmental Data Capture, Communication, and Display

(71) Applicant: ArcaneWear LLC, Seattle, WA (US)

(72) Inventor: Paul Joseph Dupuy, Redmond, WA (US)

(21) Appl. No.: 15/935,809

(22) Filed: Mar. 26, 2018

Related U.S. Application Data
(60) Provisional application No. 62/477,371, filed on Mar. 27, 2017.

(51) Int. Cl. A61B 5/00; A61B 5/024; A61B 5/0255; A61B 7/04

(52) U.S. Cl. CPC A61B 5/6822; A61B 5/02438; A61B 5/0004; A61B 5/7405; A61B 2560/0475; A61B 5/742; A61B 5/0255; A61B 7/04; A61B 5/7455

(57) ABSTRACT

The subject matter disclosed herein is directed to various articles or accessories embodied in a device having capabilities for data capture, data communication, and data display. Articles or accessories according to various embodiments may include, for example, fashion accessories such as pendants, lockets, necklaces, brooches, rings, bracelets, wristbands, and the like that may be worn by the user.

### BACKGROUND

Individuals wear fashionable accessories as a means for self-expression. A modern trend in the fashion-accessory market is designing accessories that have technological capabilities. Many of these high-tech accessories are designed to sense physiological data for the purpose of monitoring various aspects of an individual's health and fitness. Such accessories do not use the physiological or other data itself as a means for visual artistic self-expression or for sharing physiological or other data with loved ones or friends for their own personal display of self-expression through fashion.

### BRIEF DESCRIPTION OF THE DRAWINGS

Aspects and many of the attendant advantages of the claims will become more readily appreciated as the same become better understood by reference to the following detailed description, when taken in conjunction with the accompanying drawings, wherein:

FIG. 1 is a diagram of three views of a wearable device having physiological and environmental data capture capabilities according to an embodiment of the subject matter disclosed herein.

FIG. 2 is a diagram of a wearable device capturing physiological signals from a person and sound from the environment according to an embodiment of the subject matter disclosed herein.

FIG. 3 is a diagram of a wearable device interpreting electrical signals for data visualization according to an embodiment of the subject matter disclosed herein.

FIG. 4 is a diagram of a wearable device storing physiological, environmental, and related data according to an embodiment of the subject matter disclosed herein.

FIG. 5 is a diagram of a wearable device transferring physiological, environmental, and related data to a computer server and personal smartphone according to an embodiment of the subject matter disclosed herein.

FIG. 6 is a diagram of an exemplary computing system according to an embodiment of the subject matter disclosed herein.

Note that the same numbers are used throughout the disclosure and figures to reference like components and features.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

By way of overview, the subject matter disclosed herein is directed to various articles or accessories embodied in a device having capabilities for data capture, data communication, and data display. Articles or accessories according to various embodiments may include, for example, fashion accessories such as pendants, lockets, necklaces, brooches, rings, bracelets, wristbands, and the like that may be worn by the user. Further, a fashion accessory according to various embodiments may comprise one or more ornamental exterior encasements. One or more such exterior encasements may further comprise an enclosure that defines an interior cavity for housing a data capture, data communication, and data display device. The data capture, data communication, and data display device housed in an inner cavity may comprise one or more sensors and a microphone for capturing physiological and environmental data, a computing device with a touch-enabled screen for displaying captured or transmitted data and related information, a wireless communication transmitter, an internal energy source, audio speakers, a haptic actuator, and a charging connection for receiving a charging source that may include an inductive charge receiver.

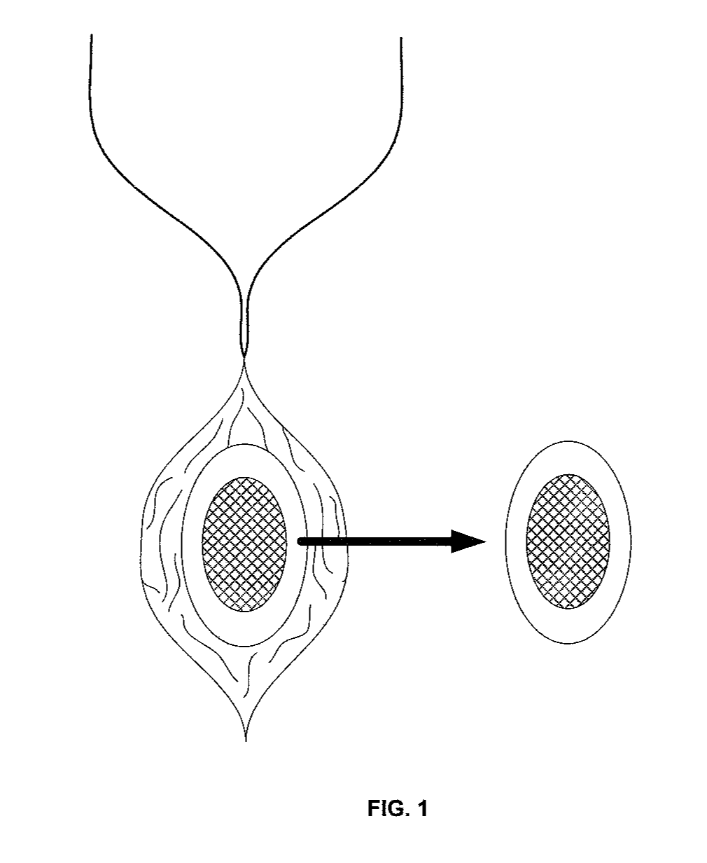

In one embodiment, the device is encased in a pendant that may be worn around a person's neck. The pendant-encased device may be capable of sensing, recording, displaying, and wirelessly transmitting and receiving physiological data such as a wearer's heartbeat and the like. To detect physiological information from the wearer of the device, the device may use touch, infrared, or ultrasonic detection sensors. To generate biometric data from the detected physiological data, the device may use a processor to interpret electric signals received from sensors. This data may then be visually displayed on the device's screen, output through the device's speaker, and applied through the device's haptic actuator. The processor may also facilitate recording and storing the captured physiological and environmental data and/or transmit that data in real-time over a wireless communication network for cloud storage or for transmission to another wirelessly-enabled display device. These and other aspects are discussed below with respect to FIGS. 1-6.

FIGS. 1-6 show features of the wearable device. In FIG. 1, this embodiment of the device is shown as encased in a pendant. FIG. 1 shows an ornamental pendant whereby a face of the housed wearable device is visible through the ornamentation of the pendant. In this embodiment, the device may be removed from within the pendant and placed into other types of ornamental encasements. The device may have a protective exterior enclosure made of a resilient and waterproof plastic or the like to protect the device's internal sensor and other electrical circuitry from direct contact with the wearer or from liquids and other elements that would degrade, damage, or impede the device's electrical circuitry.

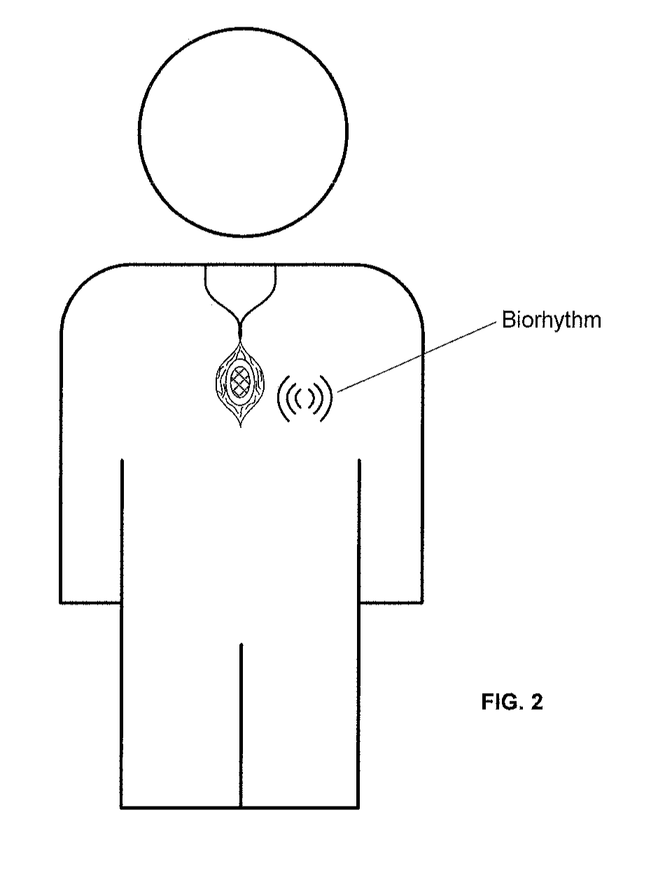

As shown in FIG. 2, when the device is engaged in an “recording” position and worn close to the body of the wearer, the physiological detection sensor may sense physiological signals of the wearer, including, for example, the wearer's heartbeat. The device may also record physiological data of the wearer or another individual by direct touch, including, for example, a person's heartbeat.

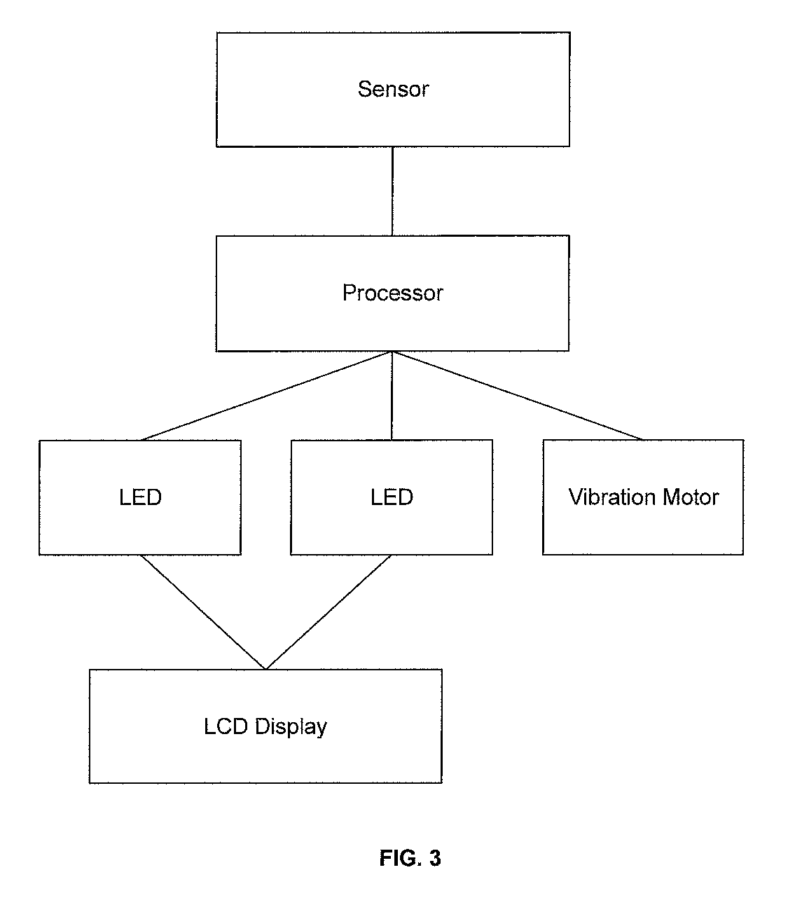

As shown in FIG. 3, once the sensors have detected physiological signals, the computing device may receive electrical signals from the detection sensors indicative of physiological processes and states and interpret the electrical signals into data that may be visualized on the device's screen and/or stored in a local memory component. In this embodiment, the screen is a Liquid Crystal Display (LCD) that includes an array of colored light emitting diodes (LEDs). The captured physiological data may be visualized using the LED array in a manner that visibly “pulses” in time with data captured from the wearer's heartbeat and may incorporate other physiological information, such as galvanic skin response data. The visual display of the data is not limited to “pulse” types, but includes a wide range of time transitional visualization techniques that incorporate data transformations of signal amplitude, frequency, duration, etc. Further, the pulses may also cause other electronic components to illuminate the pendant encasement. In another embodiment, the device may engage a vibration actuator to produce haptic expression of the physiological data, providing touch sense stimuli associated with the data in addition to, or instead of, light-assisted visualization. In another embodiment, the device may engage a speaker to produce audible feedback of the physiological data, providing auditory sense stimuli associated with the data in addition to, or instead of, light-assisted visualization and/or haptic stimulation.

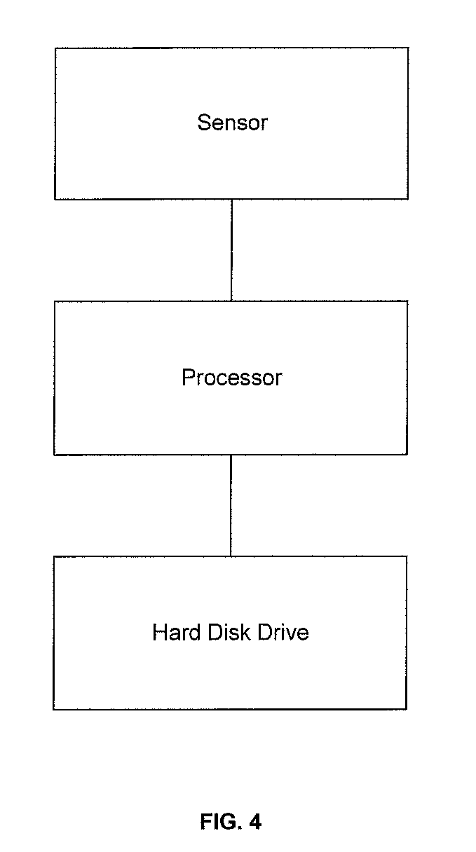

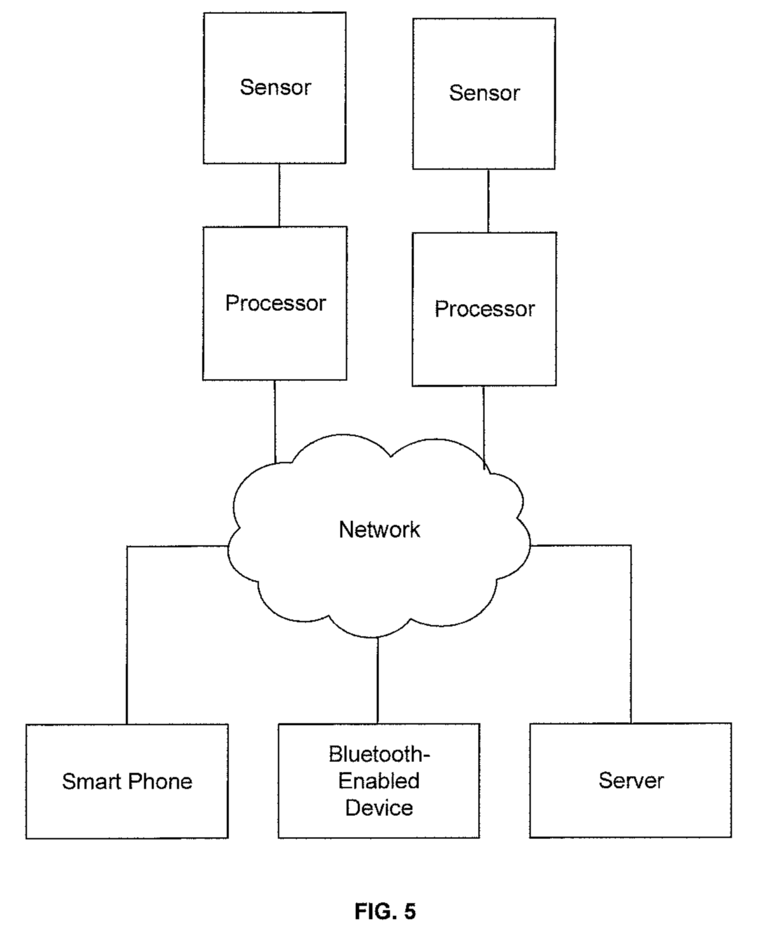

As shown in FIG. 4, although in one embodiment the device may display physiological and environmental data in real-time, in another embodiment the computing device may capture the data and store it as a file on an internal storage medium or, as shown in FIG. 5, transfer the captured data file to either a server computer or to another device capable of receiving the transmitted data file. The computing device may access the recorded data file and render it through the device's output mechanisms in the same manner as described above for real-time data visualization.

In this manner, a user of the wearable device may store and display physiological and environmental data from some other person who may be considered special or important by the wearer. For example, couples may express love for one another by recording a heartbeat to be sent to and displayed by each other. As another example, the data may be originally captured from a religious leader, such as the Pope or the Dali Lama. In this manner, the wearer may feet a greater affinity toward the individual whose data is displayed on the device.

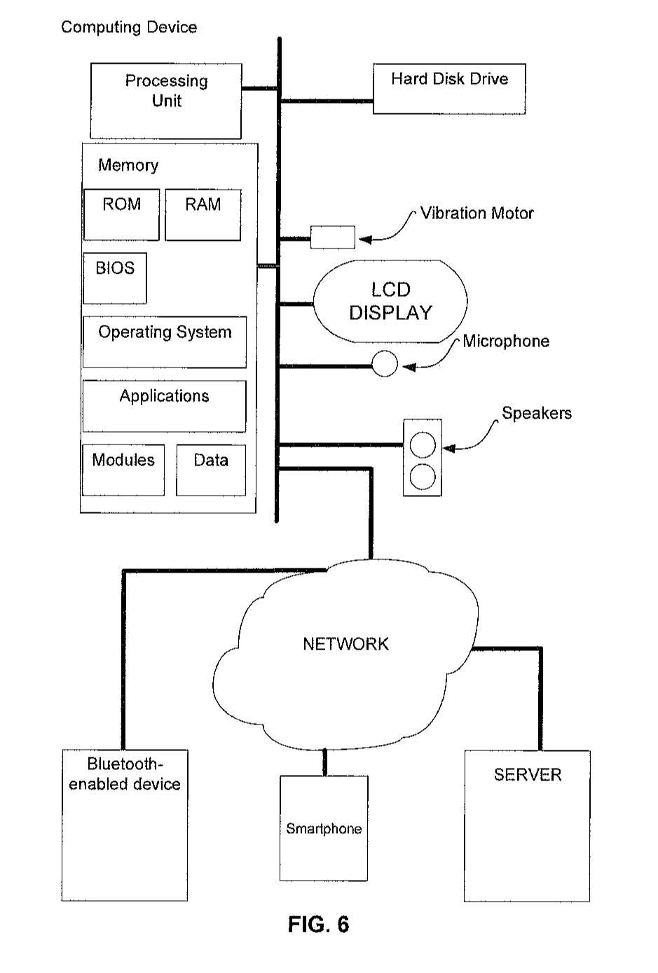

As shown in FIG. 6, an exemplary system for implementing the systems disclosed herein includes a general purpose computing device that includes a processing unit, system memory, and a system bus that couples various system components including the system memory to the processing unit. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The system memory may include read only memory (ROM) and random access memory (RAM). A basic input/output system (BIOS), containing the basic routines that help to transfer information between elements within the computing device, such as during start-up, is stored in ROM. The computing device may also include a flash drive for reading from and writing data to a storage medium. The flash drive is connected to the system bus by an appropriate interface. The drive and its associated computer-readable media provide nonvolatile storage of computer readable instructions, data structures, program modules and other data. Although the exemplary environment described herein employs a flash drive, it should be appreciated by those skilled in the art that other types of computer-readable media which can store data that is accessible by a computer, such as flash memory cards, digital versatile disks, random access memories (RAMs), read only memories (ROM), and the like, may also be used in the exemplary operating environment.

A number of program modules may be stored on the flash disk, ROM, or RAM, including an operating system, one or more application programs, other program modules, and program data. Interacting external devices may include a personal smartphone, a personal computer, a remote server, a Bluetooth-enabled paired device or the like. These and other devices are often connected to the processing unit through a wireless-communication interface, network interface, or a universal serial bus (USB). An LCD screen or other type of display may also be connected to the system bus. One or more speakers and microphones may be connected to the system bus via an interface, such as an audio adapter. A vibration actuator may be connected to the system bus via an interface and controlled by the processor.

In addition to receiving data from a variety of input devices, the computing device may transmit captured, recorded, or otherwise stored data to external devices such as a personal smartphone, a personal computer, a remote server, a Bluetooth-enabled paired device, a smart inductive charging device and the like. These other devices are often connected to the processing unit through a wireless communication interface, network interface or a USB.

In another embodiment, the computing device may be programmed to display external data received from various input devices in association with, or instead of, the real-time or recorded physiological and environmental data described above.

In another embodiment, the computing device may be capable of receiving push notifications and software updates from a variety of input devices.

In another embodiment, the wearable device's internal power source may be inductively charged in association with an inductive charging station or charged with a pluggable USB-type charging cable.

In another embodiment the computing device may employ Near Field Communication (NFC) to share captured, recorded, and/or stored data with another NFC-enabled device.

While the subject matter discussed herein is susceptible to various modifications and alternative constructions, certain illustrated embodiments thereof are shown in the drawings and have been described above in detail. It should be understood, however, that there is no intention to limit the claims to the specific forms disclosed, but on the contrary, the intention is to cover all modifications, alternative constructions, and equivalents falling within the spirit and scope of the claims.

1. An apparatus comprised of at least one exterior encasement that defines an interior cavity which houses at least one data capture device, data communication device, and data display device wherein the capture device is comprised of at least one sensor and a microphone for capturing physiological and environmental data.

2. The apparatus of claim 1 wherein the data display device displays physiological data via an audio speaker, haptic actuator, or a combination thereof.

3. The apparatus of claim 2 where physiological data is heart rate.

4. The apparatus of claim 3 where heart rate is displayed via haptic pulse.

5. The apparatus of claim 3 where heart rate is displayed via an illuminated pulse.

6. The apparatus of claim 1 where physiological data for a first user is stored on an internal storage medium or server computer.

7. The apparatus of claim 6 where a second user may request physiological data for the first user.

8. The apparatus of claim 1 where the apparatus is a fashion accessory.

9. A method to use an apparatus comprised of at least one exterior encasement that defines an interior cavity which houses at least one data capture device, data communication device, and data display device wherein the capture device is comprised of at least one sensor and a microphone for capturing physiological and environmental data includes capturing a first user's physiological data.

10. The method according to claim 9 whereby the first user's physiological data is stored on an internal storage medium or a server computer.

11. The method according to claim 10 whereby a second user requests the physiological data of the first user.

12. The method according to claim 11 where by the second user displays the first user's physiological data on apparatus comprised of at least one exterior encasement that defines an interior cavity which houses at least one data capture device, data communication device, and data display device wherein the capture device is comprised of at least one sensor and a microphone for capturing physiological and environmental data.
